(19) United States
(12) Patent Application Publication
(10) Pub. No.: US 2025/0276308 A1
(43) Pub. Date: Sep. 4, 2025

(54) LOW-TEMPERATURE OXIDATIVE DEHYDROGENATION OF LOW-CARBON ALKANES TO LIGHT OLEFINS WITH MICRO-MESOPOROUS CATALYST

(71) Applicant: Braskem America, Inc., Philadelphia, PA (US)

(72) Inventors: Ishant Khurana, Philadelphia, PA (US); Dan Xie, Philadelphia, PA (US); Graham Gregorich, Philadelphia, PA (US); Scott Mitchell, Philadelphia, PA (US)

(73) Assignee: Braskem America, Inc., Philadelphia, PA (US)

(21) Appl. No.: 19/068,863

(22) Filed: Mar. 3, 2025

Related U.S. Application Data
(60) Provisional application No. 63/560,126, filed on Mar. 1, 2024.

(51) Int. Cl. B01J 29/06; B01J 35/63; C07C 5/333

(52) U.S. Cl. CPC B01J 29/061; B01J 35/633; C07C 5/3332; C07C 2529/06

(57) ABSTRACT

The present disclosure relates to a heterogeneous catalyst composition comprising a metal catalyst chemically interacted with a micro-mesoporous aluminosilicate support. A process for catalytic oxidative dehydrogenation of hydrocarbons may include contacting, in a reactor system, a hydrocarbon-containing feedstock with the heterogeneous catalyst composition to generate olefinic compounds. A process for preparing a heterogeneous catalyst composition may include combining a micro-mesoporous aluminosilicate support with a metal catalyst precursor to form a catalyst precursor mixture, and heating the catalyst precursor mixture to a temperature of about 390° C. to about 750° C. to form a heterogeneous catalyst composition.

### BACKGROUND

Light olefins, ethylene and propylene, are important raw materials for the production of polyolefins such as polyethylene (PE) and polypropylene (PP). The increase in the demand for basic olefins and current fluctuations in their supply prices resulted in an increase in application of the on-purpose basic olefin production technologies such as the non-oxidative dehydrogenation of light alkanes. The current commercial technology offerings rely exclusively on the use of a solid catalyst that deactivates under high operating temperatures and requires frequent regeneration, either in packed into fixed bed reactors, hence limiting the process efficiency due to the need for a semi-continuous operation and having a large catalyst inventory; or packed into a moving bed, allowing for a continuous process operation, but resulting in a progressive catalyst's material loss due to mechanical abrasion and particle disintegration due to thermal shock cracking.

The commercial technologies are also highly energy- and capital-intensive, which prompted the exploration of the new process intensifying opportunities in the form of catalytic oxidative dehydrogenation (ODH). While the emerging oxidative dehydrogenation (ODH) process is thermodynamically favored and the use of gas phase oxygen leads to the continuous removal of coke deposits and to stable reaction rates, the process suffers from poor product selectivity limiting the olefin yields. Due to thermodynamic preference of secondary complete combustion at the higher temperatures generated in this highly exothermic reaction, catalysts with acceptable activity tend to over-oxidize the olefin and have unacceptably large selectivities to CO and CO₂.

Thus, the challenge in the path to process intensification lies in catalyst deactivation at high operating temperatures (>500° C.) for non-oxidative dehydrogenation and in poor product selectivity in presence of gas phase oxygen at moderate temperatures (>400° C.) for oxidative dehydrogenation. Hence, there is a need for a catalyst with superior activity that it is sustained over time, and with superior selectivity at low reaction temperatures.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to a heterogeneous catalyst composition comprising a metal catalyst chemically-interacted with a micro-mesoporous aluminosilicate support.

In another aspect, embodiments disclosed herein relate to a process for catalytic oxidative dehydrogenation of hydrocarbons that includes contacting, in a reactor system, a hydrocarbon-containing feedstock with a heterogeneous catalyst composition to generate olefinic compounds. The heterogeneous catalyst composition includes a metal catalyst chemically-interacted with a micro-mesoporous aluminosilicate support to generate an olefinic compound.

In yet another aspect, embodiments disclosed herein relate to a process for preparing a heterogeneous catalyst composition, including adding, to a micro-mesoporous aluminosilicate support, a metal catalyst precursor to form a catalyst precursor mixture; and heating the catalyst precursor mixture to a temperature of from 390° C. to 750° C. to form the heterogeneous catalyst composition. Other aspects and advantages of the claimed subject matter will be apparent from the following description and the appended claims.

### BRIEF DESCRIPTION OF DRAWINGS

FIG. 1 shows the ethylene formation rate as a function of time-on-stream during the reaction step (of two-step chemical looping process) on microporous and micro-mesoporous catalysts measured in a lab-scale plug flow reactor.

### DETAILED DESCRIPTION

Embodiments disclosed herein relate to a heterogeneous catalyst composition, a process to prepare the heterogeneous catalyst composition, and a process for oxidative dehydrogenation (ODH) of hydrocarbons to form olefinic monomers using the heterogeneous catalyst composition. In particular, embodiments described herein are directed to a heterogeneous catalyst a metal catalyst chemically-interacted with a micro-mesoporous aluminosilicate support. The heterogeneous catalyst composition may be used in a process for ODH of hydrocarbons to form olefinic monomers with improved activity which better sustained over time by using a chemical looping reactor system, while also enabling the ODH process to operate at a relatively lower temperature compared to previously reported ODH processes. Embodiments described herein also relate a process to prepare the heterogeneous composition either inside or outside a reactor system.

### Heterogeneous Catalyst Composition

One or more embodiments relates to a heterogeneous catalyst composition comprising a metal catalyst chemically-interacted with a micro-mesoporous aluminosilicate support.

The heterogeneous catalyst composition contains a metal catalyst. In one or more embodiments, the metal catalyst is a transition metal catalyst. Examples of metal catalysts may include transition metals, where the transition metal belongs to groups 5 to 12 in the Periodic Table of Elements, including but not limited to elements V, Cr, Mn, Fe, Co, Ni, Cu, Mo, Zn, W and any combination thereof, with the metal being in the oxidized state or in any transition state. More preferably, the transition metal is selected from V, Ni, Cu, Mn, or Fe, or any combination thereof, even more preferably the metal catalyst is Cu.

In one or more embodiments, the metal of the metal catalyst is in the oxidized form (i.e., one or more of its cationic forms), and it is chemically-interacted with micro-mesoporous aluminosilicate support. The term “chemically-interacted” is to be understood in this disclosure as two or more molecules or atoms in the metal catalyst and in the aluminosilicate support undergoing a chemical interaction that leads to formation of a bond. The bond can be, for example, a covalent bond, an ionic bond, a metallic bond, or a hydrogen bond. For the purposes of the present disclosure, the metal catalyst may be in the oxidized state or in any transition state.

In some embodiments, the aluminosilicate is in the form of zeolite. A zeolite is a microporous, crystalline aluminosilicate material mainly consisting of silicon, aluminum, and oxygen, typically having the general formula Mⁿ⁺_{1/n} (AlO₂)⁻(SiO₂)·yH₂O, where Mⁿ⁺_{1/n} is either a metal ion or H⁺, x is Si/Al molar ratio (or SiO₂/AlO₂ molar ratio) and is greater than 1, and y is the number of water molecules in the formula unit. Any types of zeolites well known to one skilled in the art are suitable herein as support for heterogeneous catalyst composition, provided that it is a micro-mesoporous zeolite. Exemplary types of zeolites are FAU (e.g., Zeolite X, Zeolite Y, and USY), BEA, MOR, MFI, and FER types. Typical support for the metal catalyst is an acidic support, i.e., M is H⁺. In one embodiment, the zeolite has an MFI framework, preferably a ZSM-5 zeolite. In one embodiment, the zeolite is the acidic form of ZSM-5, also referred to as H—ZSM-5.

The term “micro-mesoporous” mean that aluminosilicate support has micropores and mesopores simultaneously. For the purposes of the present invention, the term “micro-mesoporous” mean a mesopore volume of at least 0.05 cm³/g and a micropore volume of at least 0.03 cm³/g, both determined from Ar adsorption isotherms measured at 87 K.

In one or more embodiments the micro-mesoporous aluminosilicate has a mesopore volume of at least 0.04 cm³/g, for example 0.05 cm³/g, for example at least 0.10 cm³/g, for example at least 0.15 cm³/g, for example at least 0.17 cm³/g, for example at least 0.20 cm³/g, determined from Ar adsorption isotherms measured at 87 K.

In one or more embodiments, the micro-mesoporous aluminosilicate has a micropore volume of at least 0.03 cm³/g, for example at least 0.05 cm³/g, for example at least 0.07 cm³/g, for example at least 0.10 cm³/g, for example at least 0.12 cm³/g, for example at least 0.15 cm³/g, for example at least 0.17 cm³/g, for example at least 0.19 cm³/g, determined from Ar adsorption isotherms measured at 87 K.

In one or more embodiments, the metal catalyst is on the surface of the aluminosilicate, in mesopores of the aluminosilicate, in micropores of the aluminosilicate, or any combination thereof.

### Preparation of Heterogeneous Catalyst Composition

One or more embodiments disclosed herein relate to a process to prepare a heterogeneous catalyst composition. Specifically, one or more embodiments relates to preparation of a heterogeneous catalyst composition by adding, to a micro-mesoporous aluminosilicate support, a metal catalyst precursor to form a catalyst precursor mixture. Preparation of the heterogeneous catalyst composition may also include heating the catalyst precursor mixture. The catalyst precursor mixture may be heated to a temperature of from 390° C. to 750° C. to form the heterogeneous catalyst composition. The heterogeneous catalyst composition may be prepared either inside or outside a reactor system.

It is envisioned that the metal catalyst precursor mixture may include the metal to be incorporated into the catalyst (i.e., the metal catalyst) in the form of an oxide, hydroxide, metal salt or any combination thereof. In one or more embodiments, the metal salts may be selected from carbonate, nitrate, or metal salt of an organic acid, for example a metal acetate, with the metal being in any transition state.

Examples of forms taken by the metal catalyst precursor may include vanadium oxides, chromium oxides, manganese oxides, iron oxides, cobalt oxides, nickel oxides, copper oxides, molybdenum oxides, zinc oxides, tungsten oxides, vanadium carbonates, chromium carbonates, manganese carbonates, iron carbonates, cobalt carbonates, nickel carbonates, copper carbonates, molybdenum carbonates, zinc carbonates, tungsten carbonates, vanadium nitrates, chromium nitrates, manganese nitrates, iron nitrates, cobalt nitrates, nickel nitrates, copper nitrates, molybdenum nitrates, zinc nitrates, tungsten nitrates, vanadium acetates, chromium acetates, manganese acetates, iron acetates, cobalt acetates, nickel acetates, copper acetates, molybdenum acetates, zinc acetates, tungsten acetates or any combination thereof.

In one or more embodiments, the metal catalyst precursor comprises a transition metal carbonate, transition metal acetate or transition metal nitrate of one or more of V, Cr, Mn, Fe, Co, Ni, Cu, Mo, Zn and W. In one or more embodiments, the metal catalyst precursor comprises a transition metal carbonate, transition metal nitrate or transition metal acetate of Cu.

In one or more embodiments, the heterogeneous catalyst composition is prepared in a reactor system. The reactor system may include a tubular reactor, a continuous stirred tank reactor (CSTR), or a loop reactor.

In one or more embodiments, the reactor system is operated as a continuous process, a semi-continuous process, or a batch process.

In one or more embodiments, the reactor system includes a single reactor. In some embodiments, the reactor system includes at least a first reactor and a second reactor connected in a continuous loop for catalyst circulation.

In one or more embodiments, a heterogeneous catalyst composition is prepared from a micro-mesoporous aluminosilicate support and a metal catalyst precursor to form a catalyst precursor mixture. The micro-mesoporous aluminosilicate included in the process may include the micro-mesoporous aluminosilicate as previously described.

The process to prepare the heterogeneous catalyst composition may be carried out either outside or inside of the reactor system. In one or more embodiments, preparing the heterogeneous catalyst composition outside or inside the reactor system includes combining a micro-mesoporous aluminosilicate with a metal catalyst precursor to form a catalyst precursor mixture and heating the catalyst precursor mixture to a temperature of about 390° C. to about 750° C. to form the at least one heterogeneous catalyst composition. In one or more embodiments, the heating may occur at a temperature of about 450° C. to about 750° C.

In one or more embodiments, the process to prepare the heterogeneous catalyst composition is carried out outside the reactor system, and then it is loaded to the reactor system.

For the synthesis of the micro-mesoporous aluminosilicate support, any method well known to one skilled in the art is suitable herein, such as alkali treatment and surfactant template method. Examples of suitable methods for producing mesostructured aluminosilicates are described in Groen, J. C.; Moulijn, J. A.; Pérez-Ramirez, J. J. Mater. Chem. 2006, 16, 2121-2131. Desilication: on the controlled generation of mesoporosity in MFI zeolites and in US Patent Publication US 2005/0239634 A1 and are incorporated herein by reference. In one or more embodiments, the micro-mesoporous aluminosilicate support may be synthesized by hydrotreating a microporous aluminosilicate in the presence of a surfactant. The synthesis of the micro-mesoporous aluminosilicate support may be carried out in a temperature ranging from 20 to 200° C. The temperature may range from a lower limit of 20, 30, 40, 50, 60 or 70° C. and an upper limit of 200, 190, 180, 170, 160, 150, 140, 130, 120, 110 or 100° C., where any lower limit may be combined with any upper limit.

### Process for Oxidative Dehydrogenation of Hydrocarbons

One or more embodiments disclosed herein relate to a process for ODH of hydrocarbons to form olefinic compounds (also referred to as monomers) using the heterogeneous catalyst composition as previously described. The process may include contacting, in a reactor system, a hydrocarbon-containing feedstock with the heterogenous catalyst composition to generate olefinic compounds.

The reactor system used in the process for ODH of hydrocarbons to form olefinic compounds or olefinic monomers may include any of the reactor systems as previously described.

As described above, the reactor system may include a tubular reactor, a continuous stirred tank reactor (CSTR), or a loop reactor and the reactor system may be operated as a continuous process, a semi-continuous process, or a batch process. The reactor system may include a single reactor or at least a first reactor and a second reactor, where the first and second reactors are connected in a continuous loop for catalyst circulation.

In some embodiments, the reactor system includes a single reactor. In embodiments where the process for ODH of hydrocarbons to form olefinic compounds or olefinic monomers is carried out in a single reactor, the at least one heterogeneous catalyst composition is contacted sequentially: first with a hydrocarbon-containing feedstock, then with an oxygen source.

The process for ODH of hydrocarbons to form olefinic compounds may include contacting at least one heterogeneous catalyst composition with a hydrocarbon-containing feedstock. The hydrocarbon-containing feedstock may include a refinery range hydrocarbon. In one or more embodiments, the refinery range hydrocarbon includes at least one light alkane, for example C₂-C₈ alkanes, such as ethane, propane, n-butane, isobutane, n-pentane, isopentane, n-hexane, isohexane, n-heptane, n-octane and any combination thereof.

In some embodiments, the hydrocarbon-containing feedstock optionally contains a diluent. The diluent may include nitrogen (N₂), argon (Ar), or helium (He).

In one or more embodiments, the at least one heterogeneous catalyst composition is prepared outside of the reactor system. In this case, the process for catalytic oxidative dehydrogenation of hydrocarbons may include preparing the at least one heterogeneous catalyst composition outside of the reactor system and loading the at least one heterogeneous catalyst composition into the reactor system.

In one or more embodiments, the at least one heterogeneous catalyst composition is prepared inside of the reactor system. In this case, the process for catalytic oxidative dehydrogenation of hydrocarbons may include preparing the at least one heterogeneous catalyst composition may include loading a catalyst precursor mixture into the reactor system and heating at a temperature of about 390° C. to about 750° C., preferably of about 450° C. to about 750° C.

The process for ODH of hydrocarbons to form olefinic compounds may include contacting at least one heterogeneous catalyst composition with an oxygen source. In one or more embodiments, the oxygen source includes a purified oxygen (O₂) stream, an air stream, or a mixture thereof. In some embodiments, the oxygen source optionally contains a diluent. The diluent may include carbon dioxide (CO₂), nitrogen (N₂), argon (Ar), or helium (He).

In one or more embodiments, the process for ODH of a hydrocarbon feedstock to form olefinic compounds using at least one heterogeneous catalyst composition is an exothermic process. In one or more embodiments, the contacting in a reactor system is conducted at a temperature of about 750° C. or less and/or a pressure of about 5 atm or less. For example, the contacting process temperature may be in a range having a lower limit of about 0, 20, 30, 40, 50, 100, 150, or 200° C. and an upper limit of about 400, 450, 500, 550, 600, 650, 700 and 750° C., where any lower limit and the upper limit may be used in combination. Additionally, for example, the contacting process pressure may be in a range having a lower limit of about 0, 1, 2, or 3 atm and an upper limit of about 5, 4, or 3 atm, where any lower limit and the upper limit may be used in combination when feasible.

The olefinic compound produced by the process for ODH of hydrocarbons described herein may include light olefins, α-olefins, and terminal dienes. Some examples of olefinic compounds may include ethene, propene, 1-butene, 2-methyl-but-1-ene, 1-n-pentene, 1-n-hexene, 2-methyl-pent-1-ene, 3-methyl-pent-1-ene, 1,3-butadiene, 1,3-pentadiene, 1,4-pentadiene, 1,3-hexadiene, 1,4-hexadiene, or 1,5-hexadiene.

### EXAMPLES

The following example is provided for the purpose of further illustrating embodiments described herein and is in no way to be taken as limiting.

### Microporous Catalyst—Comparative Example 1

For the synthesis of the microporous version of the catalyst (hereinafter referred to as microporous catalyst), Cu was incorporated on commercial microporous ZSM-5 support (CBV8014, NH4-ZSM-5, SAR=80, Zeolyst) via solid-state ion-exchange, wherein 0.08 g of copper nitrate precursor was physically mixed with 5.92 g of H—ZSM-5 microporous zeolite and grounded in a mortar and pestle for about 15 mins or till the solid mixture turns light green or seems homogenous. This solid mixture was then heated to 600° C. (10° C./min) under the flow of air in a horizontal tube furnace and held for 6 h before cooling to room temperature.

### Micromesoporous Catalyst—Inventive Example 1

For the synthesis of the first micro-mesoporous version of the catalyst (hereinafter referred to as micro-mesoporous catalyst 1), Cu was supported on the mesoporous ZSM-5 support using the same type of solid-state ion-exchange procedure:

Firstly, to make mesoporous ZSM-5 support, 6 grams of commercial microporous ZSM-5 support (CBV8014, NH4-ZSM-5, SAR=80, Zeolyst) powder were mixed with 80 mL of 0.3M NaOH solution and 3 grams of Cetyltrimethylammonium bromide (CTAB). The mixture was stirred at room temperature for 30 minutes and then underwent hydrothermal treatment at 110° C. for 12 h. The resulting solid product was separated from the solution through filtration, followed by washing with deionized water and dried at 95° C. for 3 h. The dried sample was then calcined in a muffle furnace in the presence of air, heated to 550° C. at a rate of 1.5° C./min, and held for 5 h to remove the organic species inside the zeolite framework structure. Afterward, the products were treated with 60 mL of 1N ammonium nitrate solution at 95° C. for 2 h. The solution was cooled, decanted, and dried at 950° C. and this process was repeated three times. The powders were then calcined again in a muffle furnace in the presence of air, heated to 400° C. at a rate of 2° C./min, and held for 3 h to convert the zeolite sample from ammonium form to proton form.

Secondly, Cu was incorporated on this micro-mesoporous ZSM-5 support via solid-state ion exchange as well, wherein 0.08 g of copper nitrate precursor was physically mixed with 5.92 g of H—ZSM-5 micro-mesoporous zeolite and grounded in a mortar and pestle for about 15 mins or till the solid mixture turns light green or seems homogenous. This solid mixture was then heated to 600° C. (10° C./min) under the flow of air in a horizontal tube furnace and held for 6 h before cooling to room temperature.

### Micromesoporous Catalyst—Inventive Example 2

For the synthesis of the second micro-mesoporous version of the catalyst (hereinafter referred to as micro-mesoporous catalyst 2), Cu was supported on the mesoporous ZSM-5 support using the same type of solid-state ion-exchange procedure:

Firstly, to make mesoporous ZSM-5 support, 6 grams of commercial microporous ZSM-5 support (CBV8014, NH4-ZSM-5, SAR=80, Zeolyst) powder were mixed with 300 mL of 0.2M NaOH solution that was pre-heated to 80° C. The mixture was stirred at 80° C. for 2 hours in an oil bath. The resulting solid product was separated from the solution through filtration, followed by washing with deionized water and dried at 95° C. for 3 hours. The dried sample was treated with 90 mL of 2N ammonium nitrate solution at 95° C. for 3 hours. The solution was cooled, decanted, and dried at 95° C. and this process was repeated three times. The powders were then calcined in a muffle furnace in the presence of air, heated to 400° C. at a rate of 2° C./min, and held for 3 hours to convert the zeolite sample from ammonium form to proton form.

Secondly, Cu was incorporated on this micro-mesoporous ZSM-5 support via solid-state ion exchange as well, wherein 0.08 g of copper nitrate precursor was physically mixed with 5.92 g of H—ZSM-5 micro-mesoporous zeolite and grounded in a mortar and pestle for about 15 mins or till the solid mixture turns light green or seems homogenous. This solid mixture was then heated to 600° C. (10° C./min) under the flow of air in a horizontal tube furnace and held for 6 h before cooling to room temperature.

### Catalyst Characterization

Micropore and mesopore volumes of the catalysts were determined from Ar adsorption isotherms measured at 87 K on a Micromeritics ASAP 2020 Surface Area and Porosity Analyzer. Typically, 0.03-0.05 g of pelleted and sieved sample (nominal diameter between 180-250 μm) were degassed by heating to 120° C. (10° C./min) under vacuum (<5 μmHg) for 2 h, and then further heating to 350° C. (10° C./min) under vacuum (<5 μmHg) and holding for 9 h. Volumetric gas adsorption within micropores (cm³ g⁻¹ at STP) was estimated from analysis of semi-log derivative plots of the adsorption isotherm (∂(Vads)/∂(ln(P/P0)) vs. ln(P/P0)) to identify the micropore filling transition (first maximum) and then the end of micropore filling (subsequent minimum). Micropore volumes (cm³g⁻¹) were obtained by converting standard gas adsorption volumes (cm³ gcat⁻¹ at STP) to liquid volumes using a density conversion factor assuming the liquid density of Ar at −186° C. Micropore and mesopore volumes of the catalysts are shown in Table 1. As illustrated in the table, the micro-mesoporous catalyst shows an increase in the mesopore volume relative to the microporous catalyst.

TABLE 1
Micropore and mesopore volume measured on the
microporous and micro-mesoporous catalyst

| Sample | Vmicro (cm³g⁻¹) | Vmeso (cm³g⁻¹) |
|---|---|---|
| Comparative Example 1 | 0.200 | 0.030 |
| Microporous catalyst | | |
| Inventive Example 1 | 0.216 | 0.191 |
| Micro-mesoporous catalyst 1 | | |
| Inventive Example 2 | 0.224 | 0.463 |
| Micromesoporous catalyst 2 | | |

Selective Production of Ethylene from Ethane Reaction at Low Temperature

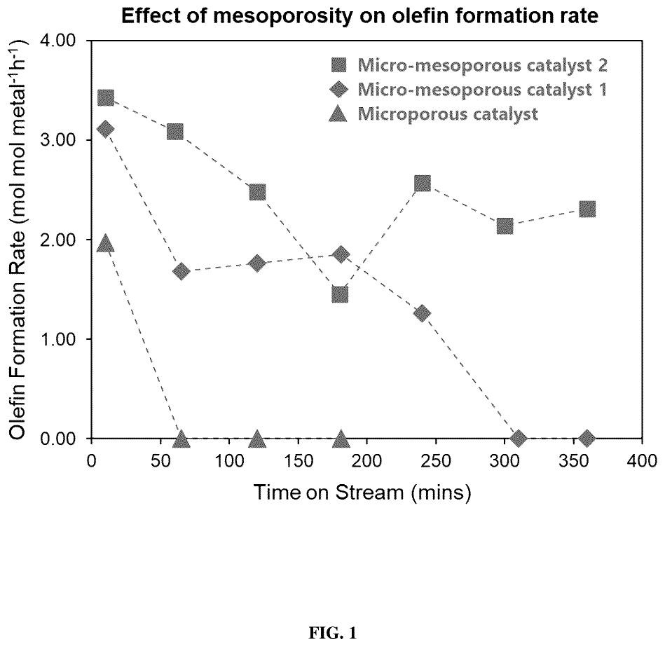

Ethane was fed onto a bed of transition metal incorporated micro-mesoporous mixed metal oxide (0.2 g) contained in a lab-scale plug flow reactor. The catalyst bed was maintained at a temperature of 350° C. Ethane feed concentration was about 35% in nitrogen and the total feed rate was about 200 ml/min. The catalyst according to the present disclosure exhibited higher initial activity and more sustained activity at 100% selectivity vs. the incumbent and conventional microporous version of the same catalyst. FIG. 1 illustrates the results comparing the ethylene formation rate as a function of time-on-stream at 350° C. on both the microporous and micro-mesoporous catalysts.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail, and a screw may be equivalent structures. It is the express intention of the applicant not to invoke 35 U.S.C. § 112(f) for any limitations of any of the claims herein, except for those in which the claim expressly uses the words ‘means for’ together with an associated function.

What is claimed:

1. A heterogeneous catalyst composition comprising a metal catalyst chemically interacted with a micro-mesoporous aluminosilicate support.

2. The heterogeneous catalyst composition of claim 1, wherein the metal catalyst is a transition metal catalyst.

3. The heterogeneous catalyst composition of claim 2, wherein the transition metal catalyst is selected from Groups 5 to 12 of the Periodic Table of Elements, and any combination thereof.

4. The heterogeneous catalyst composition of claim 1, wherein the aluminosilicate is in the form of zeolite.

5. The heterogeneous catalyst composition of claim 1, wherein the micro-mesoporous aluminosilicate has a mesopore volume of at least 0.05 cm³/g, determined from Ar adsorption isotherms measured at 87 K.

6. The heterogeneous catalyst composition of claim 1, wherein the micro-mesoporous aluminosilicate has a micropore volume of at least 0.03 cm³/g, determined from Ar adsorption isotherms measured at 87 K.

7. The heterogeneous catalyst composition of claim 1, wherein the metal catalyst is on the surface of the aluminosilicate, in mesopores of the aluminosilicate, in micropores of the aluminosilicate, or any combination thereof.

8. A process for catalytic oxidative dehydrogenation of hydrocarbons, the process comprising:
contacting, in a reactor system, a hydrocarbon-containing feedstock with a heterogeneous catalyst composition comprising a metal catalyst chemically interacted with a micro-mesoporous aluminosilicate support to generate an olefinic compound.

9. The process of claim 8, wherein the process is an exothermic process.

10. The process of claim 8, wherein contacting in the reactor system is conducted at a temperature of about 750° C. or less.

11. The process of claim 8, wherein the process is carried out at a pressure of about 5 atm or less.

12. The process of claim 8, wherein the olefinic compound comprises light olefins, α-olefins, terminal dienes, or any combination thereof.

13. The process of claim 8, wherein the hydrocarbon-containing feedstock comprises refinery range hydrocarbon.

14. The process of claim 8, wherein the contacting is in the presence of:
an oxygen source, wherein the oxygen source comprises a purified O₂ stream, an air stream, or any combination thereof; and
optionally, a diluent selected from the group consisting of nitrogen, argon, and helium.

15. The process of claim 8, wherein the reactor system comprises a single reactor or at least a first reactor and a second reactor connected in a continuous loop for catalyst circulation.

16. The process of claim 14, wherein the reactor system comprises a single reactor, and the heterogeneous catalyst composition is contacted sequentially, first with the hydrocarbon-containing feedstock, then with the oxygen source.

17. A process for preparing the heterogeneous catalyst composition of claim 1, the process comprising:
adding, to a micro-mesoporous aluminosilicate support, a metal catalyst precursor to form a catalyst precursor mixture; and
heating the catalyst precursor mixture to a temperature of from 390° C. to 750° C. to form the heterogeneous catalyst composition.

18. The process of claim 15, further comprising:
preparing the heterogeneous catalyst composition of claim 1 outside of the reactor system, wherein preparing the heterogeneous catalyst composition outside of the reactor system comprises:
adding, to a micro-mesoporous aluminosilicate support, a metal catalyst precursor to form a catalyst precursor mixture, and
heating the catalyst precursor mixture to a temperature of from 390° C. to 750° C. to form the heterogeneous catalyst composition; and
loading the heterogeneous catalyst composition into the reactor system.

19. The process of claim 14, wherein the micro-mesoporous aluminosilicate support is synthesized by hydrotreating a microporous aluminosilicate in the presence of a surfactant.

20. The process of claim 14, wherein the heating is conducted at a temperature of about 450° C. to about 750° C.
